(19) United States
(12) Patent Application Publication
(10) Pub. No.: US 2022/0079987 A1
(43) Pub. Date: Mar. 17, 2022

(54) ISOLATION AND USE OF AUTOGENOUS PLATELET DERIVED EXOSOMES

(71) Applicant: DIRECT BIOLOGICS LLC, Austin, TX (US)

(72) Inventor: Kenneth Allen PETTINE, Fort Collins, CO (US)

(21) Appl. No.: 17/420,500

(22) PCT Filed: Jan. 6, 2020
(86) PCT No.: PCT/US2020/012359

§ 371 Date: Jul. 2, 2021

Related U.S. Application Data
(60) Provisional application No. 62/788,545, filed on Jan. 4, 2019.

(51) Int. Cl. A61K 35/19; C12N 5/078

(52) U.S. Cl. CPC A61K 35/19; C12N 5/0644

(57) ABSTRACT

Disclosed are compositions and methods for the isolation of platelet derived exosomes. In some aspects the exosomes may be used to treat chronic orthopedic conditions without causing inflammation, may include extracting platelet-rich plasma (PRP) from whole blood; isolating platelets from the PRP; activating the platelets to induce the release of the exosomes; and isolating the exosomes by differential centrifugation.

This application claims the benefit of U.S. Provisional Application No. 62/788,545, filed on Jan. 4, 2019, which is incorporated herein by reference in its entirety.

### I. BACKGROUND

Platelet-rich plasma (PRP) has become increasingly popular for the treatment of many musculoskeletal injuries, but the underlying mechanism is still not fully understood. Additionally, there is contradicting evidence of the beneficial effects of PRP in treatment orthopedic and spine pathology. It important to understand that PRP is more than just platelets and that it contains many bioactive factors that act in anabolic, catabolic, proinflammatory, and anti-inflammatory pathways. The presence of leukocytes results in pro-inflammatory cellular signaling and local tissue catabolism. For example, it has been shown that leukocytes in a PRP preparation had a negative correlation with matrix synthesis and a positive correlation with matrix catabolism in tendons. Any concentration of leukocytes, therefore, is undesirable for musculoskeletal applications. If the goal of PRP is to provide balanced, yet augmented healing, any neutrophils are antagonistic to the goal. Thus, the presence of any red blood cells or their lysate remaining in the PRP preparation are proinflammatory and should be minimized or eliminated. The current methods of creating PRP all promote a localized inflammatory response, which is increasingly recognized as being the result of red blood cells, their lysate, or the presence of neutrophils in the PRP. These are pro-inflammatory and are considered to be the etiology of the unwanted effects observed with PRP treatments. To maximize the anti-inflammatory effect of PRP the presence of any leukocytes and any red blood cells or lysate should be reduced or eliminated. No current system for concentrating platelets from whole blood can accomplish this. What is needed are new methods of isolating PRPs that reduces the presence of any leukocytes and any red blood cells or lysate.

### II. SUMMARY

Disclosed are methods and compositions related to the isolation of exosomes derived from platelet rich plasma.

In one aspect, disclosed herein are methods for the isolation of platelet derived exosomes, the method comprising a) extracting platelet-rich plasma (PRP) from whole blood; b) isolating platelets from the PRP; c) activating the platelets to induce the release of the exosomes; and d) isolating the exosomes by differential centrifugation; wherein the resultant exosomes have reduced red blood cells and leukocytes relative to whole blood to allow for use in the treatment of chronic orthopedic conditions without causing inflammation.

Also disclosed herein are methods of isolating platelet derived exosomes of any preceding aspect, wherein the PRP is extracted from whole blood by density gradient centrifugation, differential centrifugation, or elutriation. For example, the PRP can be isolated from the whole blood using differential centrifugation using a centrifuge at 250×g for 15 min at least one time.

In one aspect, disclosed herein are methods of isolating platelet derived exosomes of any preceding aspect, wherein the platelets are isolated from the PRP by density gradient centrifugation, differential centrifugation, or elutriation (such as, for example, centrifugation using a centrifuge at 250×g for 15 min at least one time).

Also disclosed herein are methods of isolating platelet derived exosomes of any preceding aspect, wherein the platelets are activated by contacting the isolated platelets with CaCl₃, thrombin, collagen, lipopolysaccharide, and/or Ca²⁺ ionophores.

In one aspect, disclosed herein are methods of isolating platelet derived exosomes of any preceding aspect, wherein the exosomes are isolated by density gradient centrifugation, differential centrifugation, or elutriation (such as, for example, centrifugation in a 30% sucrose-D2O cushion and ultra-centrifuged at 100,000×g for about 70 min). In one aspect, the exosomes can be filtered prior to centrifugation to separate the platelets from the exosomes.

Also disclosed herein are methods of isolating platelet derived exosomes, wherein the number of red blood cells and leukocytes contaminating the isolated platelet rich plasma derived exosomes is at least 100, 200, 300, 400, 500, 600, 700, 800, 900, 10³, 10⁴, 10⁵, 10⁶, 10⁷, 10⁸, 10⁹, 10¹⁰-fold reduced relative to whole blood.

In one aspect, disclosed herein are platelet rich plasma derived exosome preparation made by the method of any preceding aspect and comprising 100, 200, 300, 400, 500, 600, 700, 800, 900, 10³, 10⁴, 10⁵, 10⁶, 10⁷, 10⁸, 10⁹, 10¹⁰-fold fewer red blood cells, lysate, and/or leukocytes relative to whole blood.

In one aspect, disclosed herein are methods of treating an orthopedic and spine pathology (such as, for example, osteoarthritis, injured soft tissue, degenerated disc, and/or ankylosing spondylitis) by administering to a subject a therapeutically effective amount of the isolated exosomes prepared by the method of any preceding aspect or administering to the subject a therapeutically effective amount of the exosome preparation of any preceding aspect.

### III. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Methods of Isolating Platelet Derived Exosomes

Platelet-rich plasma (PRP), which is defined by the Red Cross as plasma containing a minimum of 200,000 platelets/μL and as defined by others as requiring over 1,000,000 platelets per μL, has become increasingly popular for the treatment of orthopedic injuries and has long been known to be effective in promoting tissue repair, but the underlying mechanism is still not fully understood. Despite the prior use and current popularity, there is contradicting evidence of the beneficial effects of PRP in treatment orthopedic and spine pathology. The current methods of creating PRP all promote a localized inflammatory response, which is increasingly recognized as being the result of red blood cells, their lysate, or the presence of neutrophils in the PRP. These are pro-inflammatory and are considered to be the etiology of the unwanted effects observed with PRP treatments.

The preparation processes currently in use take advantage of differing density gradients of the components in blood to concentrate platelets. Centrifugation of whole venous blood, containing an anticoagulant, results in a plasma supernatant with a gradient of cellular concentration. Erythrocytes are the densest and will remain as the packed cell layer at the bottom of the centrifuge container. The buffy coat of white blood cells is at the top of the packed red blood cell layer. The platelets are at the highest concentration in the plasma just above the buffy coat and decrease in concentration toward the top of the plasma layer. Many systems use a 2-spin speed protocol that first reduces the number of erythrocytes and second concentrates platelets. The various systems differ in platelet collection efficiency and repeatability, final leukocyte count, platelet activation, and ease of use.

Differences between PRP preparations can be due to the propriety system selected or the many other factors that also affect the final product. Peripheral venous blood parameters influence the contents of the final PRP product. For example, platelet count in the final concentration is dependent on the whole blood platelet count in a linear manner Hematocrit also influences the final product, especially in fixed-volume separator techniques. These types of systems have a requisite volume of whole blood, which is centrifuged before removal of a predetermined volume of the packaged cells and/or plasma layer. Therefore, a variable number of erythrocytes remain in the PRP. Storage of whole blood before processing introduces additional variability of the final PRP and platelet characteristics.

There is variability in the number of platelets, red blood cells, and leukocytes in PRP that is system dependent. The leukocytes found in PRP are primarily neutrophils. The presence of leukocytes results in pro-inflammatory cellular signaling and local tissue catabolism. For example, it has been shown that leukocytes in a PRP preparation had a negative correlation with matrix synthesis and a positive correlation with matrix catabolism in tendons. Any concentration of leukocytes, therefore, is undesirable for musculoskeletal applications. If the goal of PRP is to provide balanced, yet augmented healing, any neutrophils are antagonistic to the goal. Thus, the presence of any red blood cells or their lysate remaining in the PRP preparation are proinflammatory and should be minimized or eliminated.

It is important to understand that PRP is more than just platelets and that it contains many bioactive factors that act in anabolic, catabolic, proinflammatory, and anti-inflammatory pathways. To maximize the anti-inflammatory effect of PRP the presence of any leukocytes and any red blood cells or lysate should be reduced or eliminated. No current system for concentrating platelets from whole blood can accomplish this. The methods disclosed herein address this problem.

Platelets (thrombocytes) range in size from 2 to 3 microns while circulating for 7 to 10 days at a concentration of 150 to 400×10³/μL of whole blood. They are anucleated cytoplasmic fragments of larger multinucleated progenitor cells called megakaryocytes which are located in bone marrow. Platelets are often thought of primarily for their hemostatic and coagulation functions.

The current model of platelet formation recognizes that mature megakaryocytes extend long, branching processes, designated proplatelets, which are comprise of platelet-sized swelling in tandem arrays that are connected by thin cytoplasmic bridges. These pre-platelets have been identified both in vitro and in vivo. Pre-platelet-producing megakaryocytes yield platelets that circulate in the blood.

Platelet production begins with the erosion of one pole of the megakaryocyte to generate large pseudo-podial-like structures that are elongate, thin, and branch to yield slender tubular projections of uniform diameter (2-4 μm). This is a microtubule-driven process, which unfolds over a period of 4-10 hours. Pre-platelet shafts become filled with thick bundles of hundreds of microtubules that undergo a thinning phase (to ˜20) and loop around within the proplatelet to reenter the shaft forming buds at the pre-platelet tip. Pre-platelet elongations extend outwardly at a steady rate of ˜1 μm/min and generally reach lengths of ˜0.5-1 mm Elongation/sliding of these microtubules then generate a new platelet that is released into the blood stream.

The platelet is filled with exosomes from the megakaryocyte. The exosomes contain numerous anti-inflammatory growth factors, micro and messenger RNA all from the megakaryocyte. Proteomic studies have shown that platelets contain over 800 proteins with numerous post-translational modifications, such as phosphorylation, resulting in over 1,500 protein-based bioactive factors. All of these proteins are transferred from the megakaryocyte to the platelet during the budding off process. Platelets release these growth factors and RNA via exosomes to influence target cells through a paracrine function. Platelet rich plasma (PRP) is a plasma suspension that contains all components of whole blood in varying amounts. Platelet derived exosomes (P-Es) have been demonstrated to have similar effects in wound healing as PRP. Additionally, despite the small size of P-Es, they have numerous benefits, including their ability to be released locally, their ease of travel through the body, their low immunogenicity, and the ease with which they can be obtained. In addition, it has been demonstrated that P-Es participate in molding signal transduction. To avoid the problem of contaminating red blood cells, lysate, or leukocytes, the present methods utilize exosomes derived from platelet rich plasma. Accordingly, disclosed herein are methods for the isolation of platelet derived exosomes, wherein the resultant exosomes have reduced red blood cells and leukocytes relative to whole blood to allow for use in the treatment of chronic orthopedic conditions without causing inflammation.

In one aspect, disclosed herein are methods for the isolation of platelet derived exosomes, the method comprising a) extracting platelet-rich plasma (PRP) from whole blood; b) isolating platelets from the PRP; c) activating the platelets to induce the release of the exosomes; and d) isolating the exosomes by differential centrifugation; wherein the resultant exosomes have reduced red blood cells and leukocytes relative to whole blood to allow for use in the treatment of chronic orthopedic conditions without causing inflammation.

Extraction of PRP from whole blood in the disclosed methods in one aspect comprises concentrating the platelets in the plasma and removing other cellular material. In one aspect, the method can comprise subjecting whole blood to elutriation, density gradient centrifugation (also referred to as equilibrium sedimentation), or differential centrifugation. Where centrifugation or elutriation is applied, the separation solution can comprise a 5, 6, 7, 8, 9, 10, 11, 12, 13, 14, 15, 16, 17, 18, 19, 20, 20, 25, 30, 35, 40, 45, 50, 55, 60, 65, 70% solution of Sucrose, Caesium Choloride (CsCl), percoll, Iodixanol, or Iohexol. As noted above, in one aspect, the centrifugation utilized can also be density gradient centrifugation. When density gradient centrifugation is used, the percent Sucrose, Caesium Choloride (CsCl), percoll, Iodixanol, or Iohexol increases in distinct layers increasing down the tube. In one aspect, the gradient can be any increasing amount including, but not limited to increasing layers of 5, 6, 7, 8, 9, 10, 11, 12, 13, 14, 15, 16, 17, 18, 19, 20, 20, 25, 30, 35, 40, 45, 50, 55, 60, 65, 70% solution of Sucrose, Caesium Choloride (CsCl), percoll, Iodixanol, or Iohexol.

The force applied during centrifugation can be any force appropriate for the separation of cells, organells, cellular material, proteins, and nucleic acid. For example, the force applied to the centrifugation can be at least 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 425, 450, 475, 500, 525, 550, 575, 600, 650, 700, 750, 800, 850, 900, 950, 1×10³, 1.5×10³, 2×10³, 2.5×10³, 3×10³, 3.5×10³, 4×10³, 4.5×10³, 5×10³, 5.5×10³, 6×10³, 6.5×10³, 7×10³, 7.5×10³, 8×10³, 8.5×10³, 9×10³, 9.5×10³, 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 8.5×10⁴, 9×10⁴, 9.5×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, or 1×10⁶ g. The force can be applied for any duration for the desired separation, including, but not limited to 5, 10, 15, 20, 25, 30, 35, 40, 45, 50, 55, 60, 70, 80, 90, 100, 110, 120, 135, 150, 165, 180 min, 3.5, 4, 4.5, 5, 5.5, 6, 6.5, 7, 7.5, 8, 8.5, 9, 9.5, 10, 10.5, 11, 11.5, 12, 18, 24, 36, 48 hours. Additionally, it is understood and herein contemplated that more than one spin may be needed to achieve the desired separation. In one aspect, disclosed herein are methods for the isolation of platelet derived exosomes wherein the sample is run through the elutriator or centrifuge 1, 2, 3, 4, 5, 6, 7, 8, 9, or 10 times. It is understood and herein contemplated that each run through the elutriator or centrifuge can be at a different force and/or duration.

In one aspect, the extracted platelet rich plasma comprises at least 2×10⁵, 3×10⁵, 4×10⁵, 5×10⁵, 6×10⁵, 7×10⁵, 8×10⁵, 9×10⁵, 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, or 2×10⁶ platelets per microliter.

It is understood and herein contemplated that coagulation of the whole blood during processing can occur and anticoagulants could be applied to prevent this occurrence. Thus, in one aspect, prior to centrifugation or elutriation, the method can comprise contacting the whole blood with an anticoagulant can be applied to the whole blood. For example, the method can comprise collecting whole blood from a subject and placing the whole blood into an anticoagulant (including, but not limited to, for example, acid citrate dextrose solution (ACD-A) anticoagulant, Ethylenediaminetetraacetic acid (EDTA), or oxalate). The anticoagulant can be added at a 1:50, 1:25, 1:10, 1:9, 1:8. 1:7, 1:6, 1:5, 1:4. 1:3, 1:2, or 1:1 anticoagulant to blood ratio.

For example, to extract the platelets from erythrocytes and leukocytes in plasma, about 40 mL of the mixture may be put into a 50 mL centrifuge tube and be centrifuged at 160×g for 10 min. Then, the separated plasma containing mostly platelets will be transferred to a new centrifuge tube and centrifuged at 250×g for another 15 min. Most of the supernatant plasma may be discarded before the platelet pellet may be resuspended in the residual platelet poor plasma to obtain 4 mL PRP.

Once extracted, the platelets can be isolated from the PRP via further elutriation, density gradient centrifugation (also referred to as equilibrium sedimentation), or differential centrifugation. Again, this step of elutriation or centrifugation can comprise a 5, 6, 7, 8, 9, 10, 11, 12, 13, 14, 15, 16, 17, 18, 19, 20, 20, 25, 30, 35, 40, 45, 50, 55, 60, 65, 70% solution of Sucrose, Caesium Choloride (CsCl), percoll, Iodixanol, or Iohexol. As noted above, in one aspect, the centrifugation utilized can also be density gradient centrifugation. Where density gradient centrifugation is used, the percent Sucrose, Caesium Choloride (CsCl), percoll, Iodixanol, or Iohexol increases in distinct layers increasing down the tube. In one aspect, the gradient can be any increasing amount including, but not limited to increasing layers of 5, 6, 7, 8, 9, 10, 11, 12, 13, 14, 15, 16, 17, 18, 19, 20, 20, 25, 30, 35, 40, 45, 50, 55, 60, 65, 70% solution of Sucrose, Caesium Choloride (CsCl), percoll, Iodixanol, or Iohexol.

The force applied during centrifugation can be any force appropriate for the separation of cells, organells, cellular material, proteins, and nucleic acid. For example, the force applied to the centrifugation can be at least 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 425, 450, 475, 500, 525, 550, 575, 600, 650, 700, 750, 800, 850, 900, 950, 1×10³, 1.5×10³, 2×10³, 2.5×10³, 3×10³, 3.5×10³, 4×10³, 4.5×10³, 5×10³, 5.5×10³, 6×10³, 6.5×10³, 7×10³, 7.5×10³, 8×10³, 8.5×10³, 9×10³, 9.5×10³, 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 8.5×10⁴, 9×10⁴, 9.5×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, or 1×10⁶ g. The force can be applied for any duration for the desired separation, including, but not limited to 5, 10, 15, 20, 25, 30, 35, 40, 45, 50, 55, 60, 70, 80, 90, 100, 110, 120, 135, 150, 165, 180 min, 3.5, 4, 4.5, 5, 5.5, 6, 6.5, 7, 7.5, 8, 8.5, 9, 9.5, 10, 10.5, 11, 11.5, 12, 18, 24, 36, 48 hours. Additionally, it is understood and herein contemplated that more than one spin may be needed to achieve the desired separation. In one aspect, disclosed herein are methods for the isolation of platelet derived exosomes wherein the sample is run through the elutriator or centrifuge 1, 2, 3, 4, 5, 6, 7, 8, 9, or 10 times. It is understood and herein contemplated that each run through the elutriator or centrifuge can be at a different force and/or duration. Isolating the platelets from PRP may comprise, for example, centrifuging at about 250×g for 15 min, and the platelet pellet may be washed three times with PBS (calcium-free, magnesium free, and phenol red-free).

Once isolated, the platelets can be activated to induce the release of the exosomes. Activation of the platelets can comprise contact the platelets with any agent known to induce the release of exosomes, including, but not limited to CaCl₃, thrombin, collagen, lipopolysaccharide, and/or Ca²⁺ ionophores.

To isolate the exosomes, after activation, the resuspended platelet pellet can put through an elutriation or centrifugation to remove cellular debris. The sample can be optionally filtered and subject to elutriation or centrifugation again. As with prior steps involving elutriation or centrifugation, this step of elutriation or centrifugation can comprise a 5, 6, 7, 8, 9, 10, 11, 12, 13, 14, 15, 16, 17, 18, 19, 20, 20, 25, 30, 35, 40, 45, 50, 55, 60, 65, 70% solution of Sucrose, Caesium Choloride (CsCl), percoll, Iodixanol, or Iohexol. As noted above, in one aspect, the centrifugation utilized can also be density gradient centrifugation. Where density gradient centrifugation is used, the percent Sucrose, Caesium Choloride (CsCl), percoll, Iodixanol, or Iohexol increases in distinct layers increasing down the tube. In one aspect, the gradient can be any increasing amount including, but not limited to increasing layers of 5, 6, 7, 8, 9, 10, 11, 12, 13, 14, 15, 16, 17, 18, 19, 20, 20, 25, 30, 35, 40, 45, 50, 55, 60, 65, 70% solution of Sucrose, Caesium Choloride (CsCl), percoll, Iodixanol, or Iohexol.

The force applied during centrifugation can be any force appropriate for the separation of cells, organells, cellular material, proteins, and nucleic acid. For example, the force applied to the centrifugation can be at least 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 425, 450, 475, 500, 525, 550, 575, 600, 650, 700, 750, 800, 850, 900, 950, 1×10³, 1.5×10³, 2×10³, 2.5×10³, 3×10³, 3.5×10³, 4×10³, 4.5×10³, 5×10³, 5.5×10³, 6×10³, 6.5×10³, 7×10³, 7.5×10³, 8×10³, 8.5×10³, 9×10³, 9.5×10³, 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 8.5×10⁴, 9×10⁴, 9.5×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, or 1×10⁶ g. The force can be applied for any duration for the desired separation, including, but not limited to 5, 10, 15, 20, 25, 30, 35, 40, 45, 50, 55, 60, 70, 80, 90, 100, 110, 120, 135, 150, 165, 180 min, 3.5, 4, 4.5, 5, 5.5, 6, 6.5, 7, 7.5, 8, 8.5, 9, 9.5, 10, 10.5, 11, 11.5, 12, 18, 24, 36, 48 hours. Additionally, it is understood and herein contemplated that more than one spin may be needed to achieve the desired separation. In one aspect, disclosed herein are methods for the isolation of platelet derived exosomes wherein the sample is run through the elutriator or centrifuge 1, 2, 3, 4, 5, 6, 7, 8, 9, or 10 times.

For example, the sample can be put through a series of low-speed centrifugation or elutriation steps. (300×g for 10 min, 2,000×g for 10 min) to discard cell debris. In one aspect, supernatant may be filtered through a 0.22 μm filter and transferred to a 15 mL filter unit and centrifuged at about 4,000×g. The ultra-filtered liquid may be washed with PBS three times and the ultrafiltration step may be repeated. To purify the exosomes, the ultra-filtered liquid may be transferred onto a 30% sucrose-D2O cushion and ultra-centrifuged at 100,000×g for about 70 min to pellet the exosomes, which may then be washed in a large volume of PBS and ultra-centrifuged again at the same high speed for 70 min. All centrifugations may be performed at about 4° C. The exosome pellet may be resuspended in sterile PBS and injected into the patient's arthritic joins or degenerated discs.

It is understood and herein contemplated that the disclosed methods result in isolated exosomes. The platelet rich plasma derived exosomes made by this method do not suffer from the shortcomings of other methodologies in that the end result is reduced numbers of contaminating red blood cells, lysate, and/or leukocytes relative to whole blood. In one aspect, disclosed herein are methods of isolating platelet derived exosomes, wherein the number of red blood cells, lysates, and leukocytes contaminating the isolated platelet rich plasma derived exosomes is at least 100, 200, 300, 400, 500, 600, 700, 800, 900, 10³, 10⁴, 10⁵, 10⁶, 10⁷, 10⁸, 10⁹, 10¹⁰-fold reduced relative to whole blood. It is understood that in some aspect, to determine the fold reduction in contaminating red blood cells, lysates, and leukocytes the method can comprise measuring the number of red blood cells, lysates, and leukocytes in the whole blood and in the isolated exosomes.

The disclosed methods are intended to result in a purified exosome preparation. Accordingly, disclosed herein are platelet rich plasma derived exosome preparation made by the method of any preceding aspect and comprising 100, 200, 300, 400, 500, 600, 700, 800, 900, 10³, 10⁴, 10⁵, 10⁶, 10⁷, 10⁸, 10⁹, 10¹⁰-fold fewer red blood cells, lysate, and/or leukocytes relative to whole blood.

It is understood and herein contemplated that exosomes may be beneficial for use in orthopedic treatments due to containing and releasing concentrated levels of platelet-derived growth factor (PDGF), transforming growth factor-β1 (TGF-β1), vascular endothelial growth factor (VEGF), and insulin-like growth factor (IGF), all of which are known to have beneficial effects on inflammation. In one aspect, disclosed herein are methods of treating an orthopedic and spine pathology (such as, for example, osteoarthritis, injured soft tissue, degenerated disc, and/or ankylosing spondylitis) by administering to a subject a therapeutically effective amount of the isolated exosomes prepared by any method disclosed herein or administering to the subject a therapeutically effective amount of the exosome preparation disclosed herein.

1. A method of isolating platelet derived exosomes, the method comprising:
a) extracting platelet-rich plasma (PRP) from whole blood;
b) isolating platelets from the PRP;
c) activating the platelets to induce the release of the exosomes; and
d) isolating the exosomes.
wherein the resultant exosomes have reduced red blood cells and leukocytes relative to whole blood to allow for use in the treatment of chronic orthopedic conditions without causing inflammation.

2. The method of isolating platelet derived exosomes of claim 1, wherein the PRP is extracted by density gradient centrifugation, differential centrifugation, or elutriation.

3. The method of isolating platelet derived exosomes of claim 1, wherein to extract the PRP from the whole blood, the whole blood is centrifuged at 250×g for 15 min at least one time.

4. The method of isolating platelet derived exosomes of claim 1, wherein the extracted platelet rich plasma comprises at least 2×10⁵, 3×10⁵, 4×10⁵, 5×10⁵, 6×10⁵, 7×10⁵, 8×10⁵, 9×10⁵, 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, or 2×10⁶ platelets per microliter.

5. The method of isolating platelet derived exosomes of claim 1, wherein the platelets are isolated from the PRP by applying the PRP to a centrifuge at 250×g for 15 min at least one time.

6. The method of isolating platelet derived exosomes of claim 1, wherein the isolated platelets are activated by contacting the isolated platelets with CaCl₃, thrombin, collagen, lipopolysaccharide, and/or Ca²⁺ ionophores.

7. The method of isolating platelet derived exosomes of claim 1, wherein the released exosomes are separated from the platelets by filtration prior to isolating the exosomes.

8. The method of isolating platelet derived exosomes of claim 1, wherein the exosomes are isolated by centrifugation with a 30% sucrose-D2O cushion and ultra-centrifuged at 100,000×g for about 70 min.

9. The method of isolating platelet derived exosomes of claim 1, wherein the wherein the number of red blood cells and leukocytes contaminating the isolated platelet rich plasma derived exosomes is at least 100, 200, 300, 400, 500, 600, 700, 800, 900, 10³, 10⁴, 10⁵, 10⁶, 10⁷, 10⁸, 10⁹, 10¹⁰-fold reduced relative to whole blood.

10. A platelet rich plasma derived exosome preparation made by the method of claim 1 and comprising 100, 200, 300, 400, 500, 600, 700, 800, 900, 10³, 10⁴, 10⁵, 10⁶, 10⁷, 10⁸, 10⁹, 10¹⁰-fold fewer red blood cells, lysate, and/or leukocytes relative to whole blood.

11. A method of treating an orthopedic and spine pathology by administering to a subject a therapeutically effective amount of the isolated exosomes prepared by the method of claim 1 or administering to the subject a therapeutically effective amount of the exosome preparation of claim 10.

12. The method of treating an orthopedic and spine pathology of claim 11, wherein the orthopedic or spine pathology comprises osteoarthritis, injured soft tissue, degenerated disc, or ankylosing spondylitis.
